(19) United States
(12) Patent Application Publication
(10) Pub. No.: US 2015/0262781 A1
(43) Pub. Date: Sep. 17, 2015

(54) METHOD FOR PRODUCING A STABLE AND REPRODUCIBLE ELECTRON GUN EMISSION

(71) Applicant: Paul H. Leek, Willimantic, CT (US)

(72) Inventor: Paul H. Leek, Willimantic, CT (US)

(21) Appl. No.: 14/212,684

(22) Filed: Mar. 14, 2014

(51) Int. Cl. H01J 29/98; A61N 5/10; H05H 7/02

(52) U.S. Cl. CPC H01J 29/98; H05H 7/02; A61N 5/1077; A61N 2005/1089

(57) ABSTRACT

A method for producing a stable and reproducible electron gun emission and a system for producing high energy electrons, which includes an RF source and RF components including an electron gun that is powered by a DC power supply are provided. The inventive method and system are particularly advantageous for medical radiation treatment applications.

### TECHNICAL FIELD

The present invention generally relates to a method for producing a stable and reproducible electron gun emission, and more particularly relates to the use of a direct current power supply for an electron gun.

### BACKGROUND AND SUMMARY OF THE INVENTION

Electron guns have been used for decades to provide a source of electrons for devices such as linear accelerators. For example, electron guns can be used as a source of charged particles for linear accelerators used in medical radiation treatment, radiation processing of materials and other applications such as basic or applied research. For many applications, the electron gun is used to generate charged particles for input to a radio frequency (RF) accelerator waveguide. The accelerator waveguide receives the input charged particles and accelerates them to produce an accelerated output beam of a desired frequency for use in a particular application.

Electron guns used on accelerators for medical radiation treatment applications are operated at very low beam current as the ionizing effectiveness of an electron beam is much higher than an x-ray beam.

Arcs and power excursions that occur in RF power circuits (e.g., magnetron circuits) for these accelerators or RF sources, however, adversely affect the stability and reproducibility of the gun emission. In fact, unexpected high currents have proven fatal to patients undergoing medical radiation treatment using these electron linear accelerators.

The present invention addresses this drawback by providing a method for producing a stable and reproducible electron gun emission, the method comprising using a direct current (DC) power supply for the electron gun.

In an exemplary embodiment of the inventive method, the electron gun is used as a source of electrons for a linear accelerator used in medical radiation treatment.

The present invention also provides a system for producing high energy electrons, which comprises an RF source (i.e., an electron accelerator structure defining an electron flow path and having an electron injection end and an electron exit end), and RF components including an RF power or driving source for the accelerator and an electron gun having an electron source, which is located at the injection end of the electron accelerator structure, for producing and delivering a stream of electrons to the accelerator structure, wherein the electron gun is powered by a DC power supply.

In an exemplary embodiment, the system is used in medical radiation treatment. In this exemplary embodiment, the RF power or driving source is a magnetron (due to its smaller size) and the DC power supply operates at a voltage less than 35 kilovolts, preferably less than or equal to about 5 kilovolts.

In a preferred embodiment, the RF components further include one or more circulators, one or more resisters, and one or more isolation transformers.

Other features and advantages of the invention will be apparent to one of ordinary skill from the following detailed description and accompanying drawing.

Unless otherwise defined, all technical and scientific terms used herein have the same meaning as commonly understood by one of ordinary skill in the art to which this invention belongs. All publications, patent applications, patents and other references mentioned herein are incorporated by reference in their entirety. In case of conflict, the present specification, including definitions, will control. In addition, the materials, methods, and examples are illustrative only and not intended to be limiting.

### BRIEF DESCRIPTION OF THE DRAWINGS

The present disclosure may be better understood with reference to the following drawing (FIG. 1). Components in the drawing are not necessarily to scale, emphasis instead being placed upon clearly illustrating the principles of the present disclosure.

FIG. 1 is a simplified schematic plan view of an exemplary embodiment of the inventive system, where the RF components for the RF based linear accelerator include an electron gun for producing and delivering a stream of electrons to the linear accelerator, and a DC power supply for powering the electron gun.

### DETAILED DESCRIPTION OF THE INVENTION

By way of the present invention, the possibility of significantly higher current in the event of an electrical arc or power excursion during operation of a system for producing high energy electrons is reduced or eliminated by the use of a DC power supply for the electron gun.

As mentioned above, the inventive system for producing high energy electrons comprises an RF source or electron accelerator structure, and RF components including an electron gun that is powered by a DC power supply.

In an exemplary embodiment, the system is used in radiation therapy. In this exemplary embodiment, the RF source is a so-called medical linac that produces electrons with an energy range from 4 MeV up to around 25 MeV. The shape and intensity of the beam produced by such a medical linac may be modified or collimated by a variety of means.

Preferred medical linacs include 10 MeV electron beam systems, which have a very low duty cycle and low current to provide several hundred Rads per minute of electron beam. These preferred linacs are capable of variable electron beam energy to vary the depth of tissue radiated. As is known, electrons have limited penetration in tissue, from about 1.75 to about 2 inches for 10 MeV.

Also included among the preferred medical linacs are multi-energy systems, which allow the user to choose a suitable energy up to 10 MeV for treatment.

In this exemplary embodiment, a magnetron, due to its smaller size, is preferably used as the driving force for the medical linac, and the electron gun is operated at a voltage less than 35 kilovolts, preferably less than or equal to about 5 kilovolts.

In a preferred embodiment, the DC power supply is used in conjunction with a bias power supply and the RF components further include one or more circulators to shunt power, one or more resisters for applying a resistor-divided voltage to electrode elements provided in the electron gun circuit, and one or more isolation transformers to drive the electron gun. In this preferred embodiment, the gun pulse current is less than 10 milliamps.

This preferred embodiment needs a very low current (i.e., a few milliamps of beam current). So using a DC power supply even at several kilovolts uses only a few watts. Tests have shown that a gun drive voltage of as low as 2.5 kilovolts with a specially graduated accelerating cavity design accepts about 20 percent (20%) of gun current. So for an electron beam of 2 to 10 milliamps, which is typical, the gun current required is 10 to 50 milliamps. A gun drive power supply at 5 kilovolts and 50 milliamps is only 250 watts. This power supply operates continuously during operation. It can be switched on and off in tandem with the modulator to reduce power loss, but the supply does not need to be pulsed. This allows a control system to easily monitor the gun voltage and current. By operating at a stable, low voltage the gun can be isolated from the arcs and power excursions that occur in the magnetron circuit. This makes the gun emission very stable and reproducible.

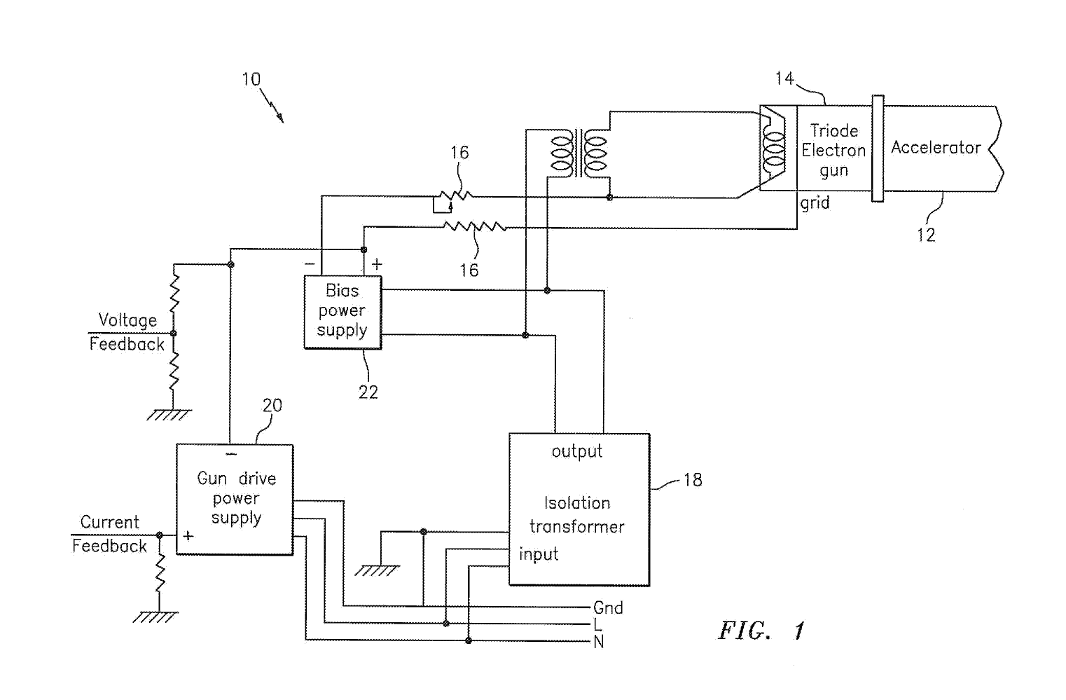

Referring now to the drawing in detail, an exemplary embodiment of the inventive system is shown in FIG. 1, marked with reference numeral 10. System 10 is made up of electron beam producing means in the form of an RF based linear accelerator or linac 12 and RF components, namely, a magnetron (not shown), electron gun 14, resistors 16, and isolation transformer 18, and serves to generate a very low electron beam current of from about 2 to about 10 milliamps. System 10 may also include in-line monitors (not shown) for monitoring gun voltage and gun current. In this preferred embodiment, the gun drive power supply 20, which is used in conjunction with a gun bias power supply 22, can be anything up to several kilovolts; 5 kilovolts can be considered typical. The gun bias power supply 22 is a few volts. This can be a positive or negative bias depending on the gun characteristics and the gun drive voltage chosen. The inventive system has been tested with a gun drive of 2.5 kilovolts and bias supply at 50 volts. The standing gun current is limited to 10 milliamps by the gun drive power supply and the cathode resistors 16.

While various embodiments of the present invention have been described above, it should be understood that they have been presented by way of example only, and not limitation. Thus, the breadth and scope of the present invention should not be limited by any of the exemplary embodiments.

We claim:

1. A method for producing a stable and reproducible electron gun emission, the method comprising using a direct current power supply for an electron gun that is used as a source of electrons for a linear accelerator.

2. The method of claim 1, wherein the linear accelerator is used in medical radiation treatment.

3. A system for producing high energy electrons, which comprises an electron accelerator structure defining an electron flow path and having an electron injection end and an electron exit end, and radio frequency components including a radio frequency power or driving source for the accelerator and an electron gun having an electron source, which is located at the injection end of the electron accelerator structure, for producing and delivering a stream of electrons to the accelerator structure, wherein the electron gun is powered by a direct current power supply.

4. The system of claim 3, which is used in medical radiation treatment, wherein the radio frequency power or driving source is a magnetron, and wherein the direct current power supply operates at a voltage less than 35 kilovolts.

5. The system of claim 4, wherein the direct current power supply operates at a voltage less than or equal to about 5 kilovolts.

6. The system of claim 4, wherein the direct current power supply is used in conjunction with a bias power supply and the radio frequency components further include one or more circulators, one or more resisters, and one or more isolation transformers.

7. The system of claim 6, which further includes in-line monitors for monitoring gun voltage and gun current.
